(19) United States
(12) Patent Application Publication
(10) Pub. No.: US 2013/0337040 A1
(43) Pub. Date: Dec. 19, 2013

(54) COMBINATION OF CERAMIDE AND OXALIPLATIN FOR INDUCING CELL DEATH AND USES THEREOF IN TREATING CANCER

(71) Applicant: Harold J. Wanebo, Bristol, RI (US)

(72) Inventor: Harold J. Wanebo, Bristol, RI (US)

(21) Appl. No.: 13/730,659

(22) Filed: Dec. 28, 2012

Related U.S. Application Data
(63) Continuation of application No. 11/809,418, filed on May 31, 2007, now abandoned.
(60) Provisional application No. 60/810,243, filed on Jun. 2, 2006.

(51) Int. Cl. A61K 31/555; A61K 31/164

(52) U.S. Cl. CPC A61K 31/555; A61K 31/164
USPC 424/450; 514/492

(57) ABSTRACT

This invention provides a method for increasing apoptosis in a cancer cell comprising contacting the cancer cell with (a) oxaliplatin and (b) C6-ceramide, sequentially or concomitantly, wherein the oxaliplatin and C6-ceramide are in amounts such that the apoptosis induced by the combination of oxaliplatin and C6-ceramide is greater than the apoptosis induced by contacting the cancer cell with either oxaliplatin alone or C6-ceramide alone. This invention also provides a method of decreasing the size of a tumor, which method comprises contacting the tumor with (a) oxaliplatin and (b) C6-ceramide, sequentially or concomitantly, wherein the oxaliplatin and C6-ceramide are in amounts such that the decrease in tumor size induced by the combination of oxaliplatin and C6-ceramide is greater than the decrease in tumor size induced by contacting the tumor with either oxaliplatin alone or C6-ceramide alone.

Throughout this application, various publications are referenced. Full bibliographic citations for these publications are found at the end of the specification immediately preceding the claims. The disclosures of these publications in their entireties are hereby incorporated by reference into this application in order to more fully describe the state of the art known to those skilled therein as of the date of the invention described and claimed herein.

### BACKGROUND OF THE INVENTION

Sphingomyelin, a cell membrane component, can be hydrolyzed to ceramide and phosphorylcholine by acid or neutral sphingomyelinase (1,2). This hydrolysis event initiates an intracellular signalling cascade associated with the stimulation of numerous biological activities, including induction of apoptosis (3-10) and arrest of cell growth in the G₀-G₁ phase (11-13).

Sphingolipids have been shown to be biologically active and have numerous regulatory effects on cell function including cell growth and differentiation. A number of inducers of sphingomyelin hydrolysis causing concommitant elevation of intracellular ceramide have been identified. These include TNFα, endotoxins, interferon α, IL-1, Fas ligand, CD28, chemotherapeutic agents, heat and ionizing radiation (14, 15). The kinetics of endogenous ceramide formation and accumulation appear to be complex and variable in different cell systems and with different inducers of sphingomyelin catabolism (16-19). It has recently been established that endogenously generated ceramide acts as a second messenger and induces apoptosis (20). Ceramide synthesis de novo has been implicated in lethal responses to several chemotherapeutic agents such as anthracyclines (21) and ara-C (22). Many recent studies have examined the effect of exogenous ceramide on the induction of apoptosis in a variety of tumor cells. Ceramide has been shown in such cases to cause cell cycle arrest in several cell lines as well as apoptosis, cell senescence and terminal differentiation (23-26). Exogenous addition of ceramide has been shown to cause apoptosis in a variety of tumor cell lines (23, 30).

Ceramide (C6-ceramide) is an analog of endogenous ceramides, which are a major signaling pathway for apoptosis in cells undergoing stress or exposure to chemotherapy.

### SUMMARY OF TEE INVENTION

This invention provides a method for increasing apoptosis in a cancer cell comprising contacting the cancer cell with (a) oxaliplatin and (b) C6-ceramide, sequentially or concomitantly, wherein oxaliplatin and C6-ceramide are in amounts such that the apoptosis induced by the combination of oxaliplatin and C6-ceramide is greater than the apoptosis induced by contacting the cancer cell with either oxaliplatin alone or C6-ceramide alone, thereby increasing apoptosis in the cancer cell.

This invention also provides a method of decreasing the size of a tumor, wherein the tumor comprises cancer cells, which method comprises contacting the tumor with (a) oxaliplatin and (b) C6-ceramide, sequentially or concomitantly, wherein oxaliplatin and C6-ceramide are in amounts such that the decrease in tumor size induced by the combination of oxaliplatin and C6-ceramide is greater than the decrease in tumor size induced by contacting the tumor with either oxaliplatin alone or C6-ceramide alone, thereby decreasing the size of the tumor.

This invention provides a pharmaceutical composition comprising oxaliplatin, C6-ceramide and a pharmaceutically acceptable carrier, wherein (i) the composition causes apoptosis in a cancer cell, and (ii) the apoptosis induced by the combination of oxaliplatin and C6-ceramide is greater than the apoptosis induced by contacting the cancer cell with either oxaliplatin alone or C6-ceramide alone.

This invention provides a method for treating a subject afflicted with cancer which method comprises administering to the subject (a) oxaliplatin and (b) C6-ceramide, sequentially or concomitantly, wherein oxaliplatin and C6-ceramide are in amounts such that the apoptosis in the subject's cancer cells induced by the combination of oxaliplatin and C6-ceramide is greater than the apoptosis in the subject's cancer cells induced by contacting the cancer cells with either oxaliplatin alone or β6-ceramide alone, thereby treating the subject afflicted with cancer.

### BRIEF DESCRIPTION OF THE FIGURES

FIG. 1

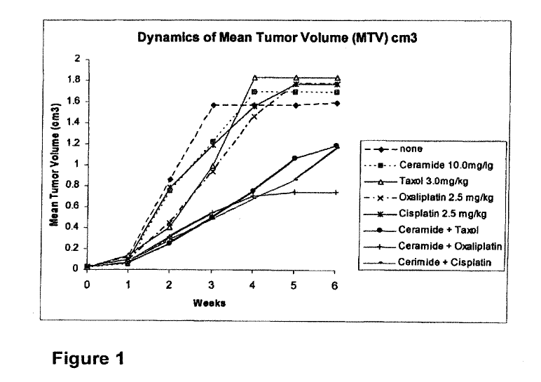

This Figure shows the dynamics of Mean Tumor Volume (MTV) [measured in cm³] for mice having been administered taxol (3.0 mg/kg), oxaliplatin (2.5 mg/kg), cisplatin (2.5 mg/kg), ceramide (10.0 mg/kg) or combinations thereof, i.e. ceramide+taxol, ceramide+oxaliplatin or ceramide+cisplatin. [Legend: control (none)=♦; taxol=Δ; oxaliplatin=x; cisplatin=*; ceramide=▪; ceramide+taxol=•; ceramide+oxaliplatin=+; and ceramide+cisplatin=−].

FIG. 2

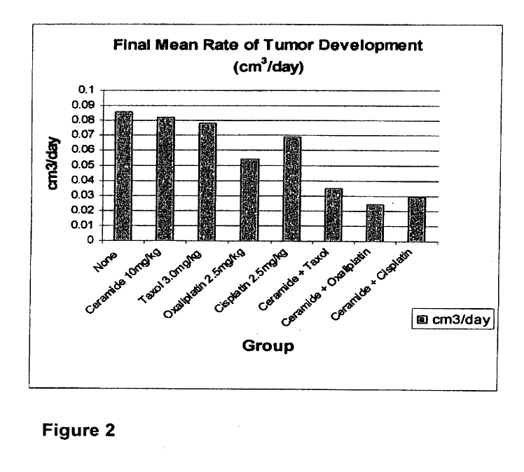

This Figure shows Final Mean Rate of Tumor Development (cm³/day) for mice having been administered taxol (3.0 mg/kg), oxaliplatin (2.5 mg/kg), cisplatin (2.5 mg/kg), ceramide (10.0 mg/kg) or combinations thereof, i.e. ceramide+taxol, ceramide+oxaliplatin or ceramide+cisplatin, or control mice not administered either ceramide or any chemotherapeutic agent (none).

FIG. 3

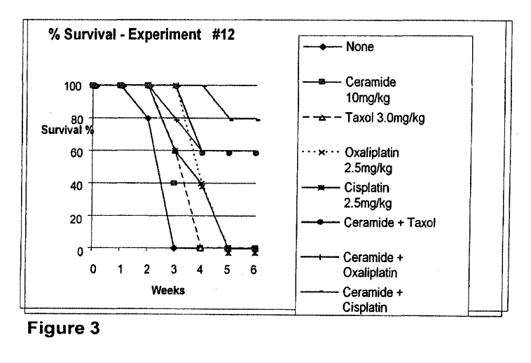

This Figure shows the percent survival over a six week period of mice having been administered taxol (3.0 mg/kg), oxaliplatin (2.5 mg/kg), cisplatin (2.5 mg/kg), ceramide (10.0 mg/kg) or combinations thereof, i.e. ceramide+taxol, ceramide+oxaliplatin or ceramide+cisplatin, or control mice not administered either ceramide or any chemotherapeutic agent (none). [Legend: control (none)=♦; taxol=Δ; oxaliplatin=x; cisplatin=*; ceramide=▪; ceramide+taxol=•; ceramide+oxaliplatin=+; and ceramide+cisplatin=−].

FIG. 4

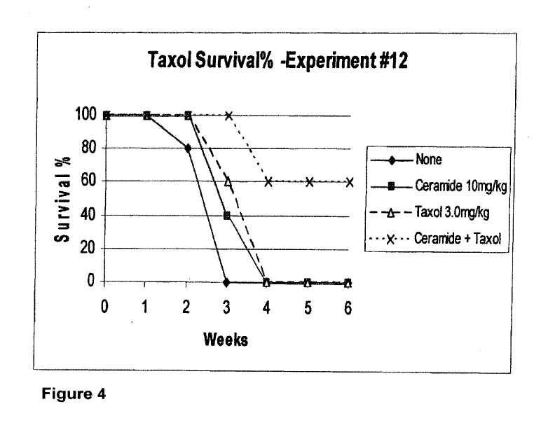

This Figure shows percent taxol survival over a six week period of mice having been administered taxol (3.0 mg/kg), ceramide (10.0 mg/kg), ceramide+taxol, or control mice not administered either ceramide or taxol (none). [Legend: control (none)=♦; taxol=Δ, ceramide=▪; ceramide+taxol=x].

FIG. 5

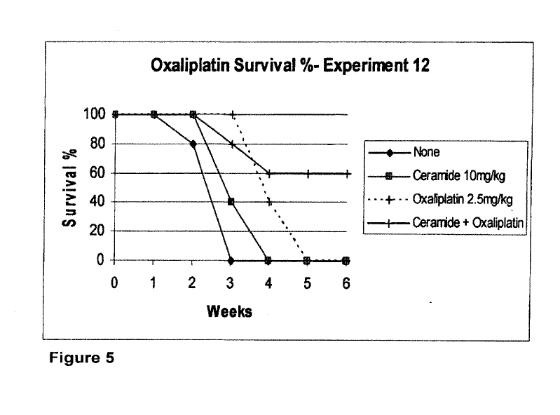

This Figure shows percent oxaliplatin survival over a six week period of mice having been administered oxaliplatin (2.5 mg/kg), ceramide (10.0 mg/kg), ceramide+oxaliplatin, or control mice not administered either ceramide or oxaliplatin (none). [Legend: control (none)=♦; oxaliplatin=- - + - -; ceramide+oxaliplatin= [symbol] ].

FIG. 6

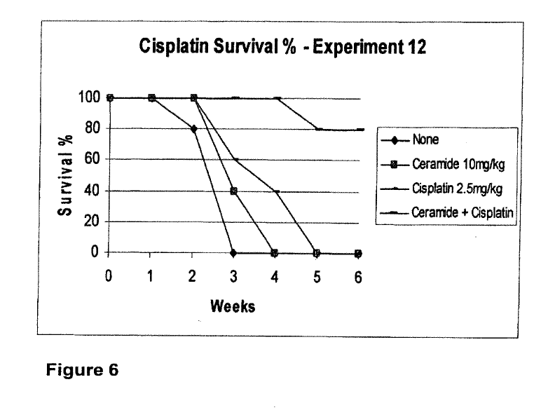

This Figure shows percent cisplatin survival over a six week period of mice having been administered cisplatin (2.5 mg/kg), ceramide (10.0 mg/kg), ceramide+cisplatin, or control mice not administered either ceramide or cisplatin (none). [Legend: control (none)=♦; cisplatin=−; and ceramide+cisplatin=−].

FIG. 7

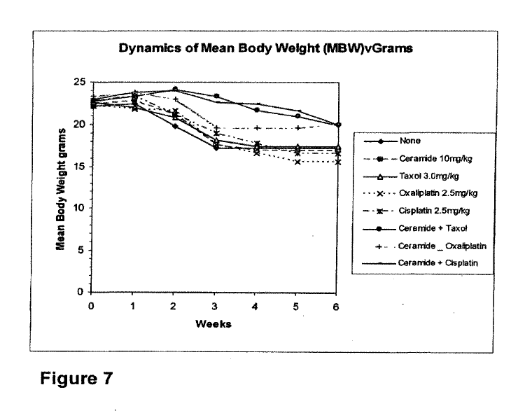

This Figure shows Mean Body Weight (MBW) over a six week period of mice having been administered taxol (3.0 mg/kg), oxaliplatin (2.5 mg/kg), cisplatin (2.5 mg/kg), ceramide (10.0 mg/kg) or combinations thereof, i.e. ceramide+taxol, ceramide+oxaliplatin or ceramide+cisplatin, or control mice not administered either ceramide or any chemotherapeutic agent (none). [Legend: control (none)=♦; taxol=Δ; oxaliplatin=x; cisplatin=*; ceramide=ceramide+taxol=•; ceramide+oxaliplatin=+; and ceramide+cisplatin=−].

FIG. 8

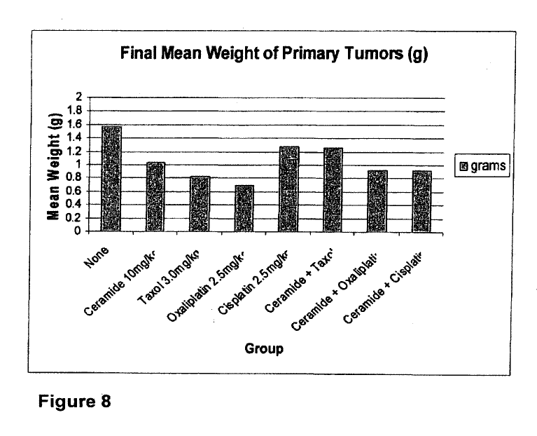

This Figure shows the Final Mean Weight of Primary Tumors (g) from mice having been administered taxol (3.0 mg/kg), oxaliplatin (2.5 mg/kg), cisplatin (2.5 mg/kg), ceramide (10.0 mg/kg) or combinations thereof, i.e. ceramide+taxol, ceramide+oxaliplatin or ceramide+cisplatin, or control mice not administered either ceramide or any chemotherapeutic agent (none).

FIG. 9

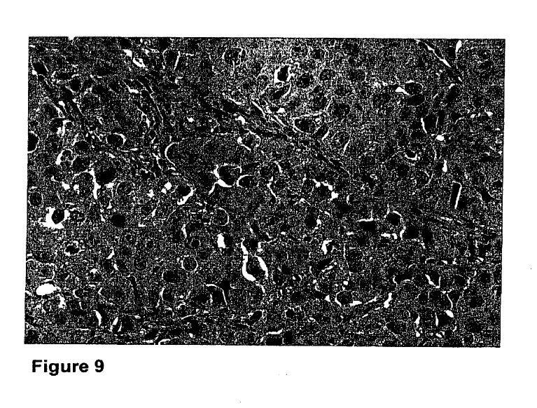

This Figure shows a demonstration of apoptosis of L3-6 pancreatic cancer using paclitaxel and ceramide (H&E examination).

FIG. 10

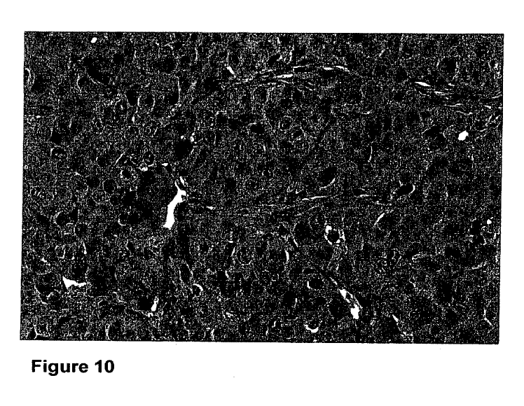

This Figure shows a demonstration of apoptosis of L3-6 pancreatic cancer in controls (H&E examination).

FIG. 11

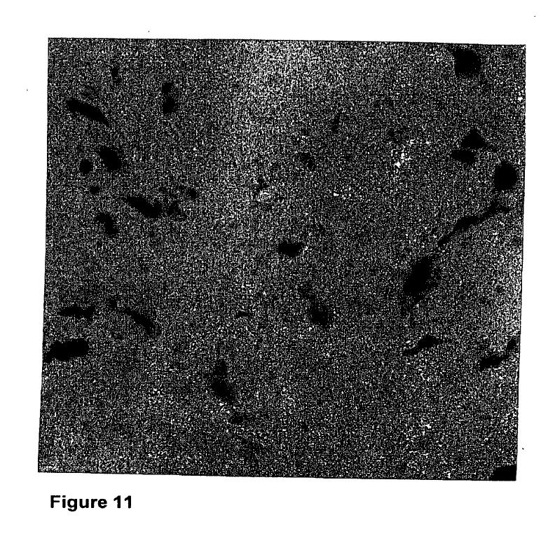

This Figure shows Caspase 3 expression after combined therapy with paclitaxel and ceramide.

FIG. 12

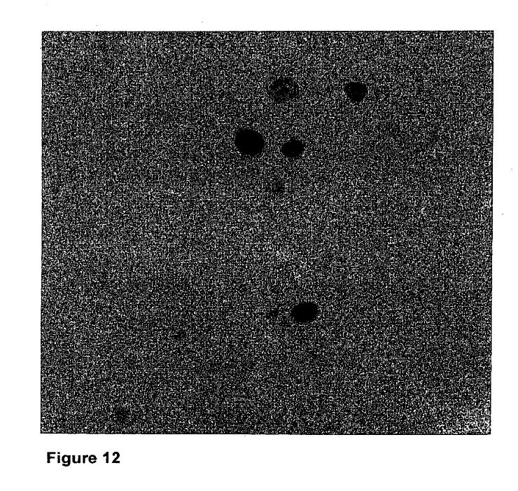

This Figure shows Caspase 3 expression in controls.

### DETAILED DESCRIPTION OF THE INVENTION

### Definitions

As used in this application, except as otherwise expressly provided herein, each of the following terms shall have the meaning set forth below.

As used herein a “ceramide” is any N-acylsphingosine. Ceramides include sphingolipids in which the sphingosine is acylated with a fatty acid acyl CoA derivative to form an N-acylsphingosine. Ceramide may be either naturally occurring or chemically synthesized. Preferably, the carbon chain length is less than 18 carbons. Examples include C6-ceramide (N-hexanoyl-D-sphingosine), C2-ceramide (N-acetyl-D-sphingosine), C8-ceramide (N-octyl-o-sphingosine) and C16-ceramide (N-palmitoyl-o-sphingosine. Other ceramides are known to one of skill in the art. Preferably, the ceramide (which is lipid soluble) is water soluble or made water soluble to enable contact with the cancer cells in a subject. Ceramide (6%) may be solubilized initially in alcohol and then subsequently diluted in saline or a cremophore.

As used herein “contacting cancer cells” is defined as exposing the cancer cells to combination therapy, i.e. administering to the cancer cells directly or indirectly, oxaliplatin and ceramide by local, regional or systemic means.

As used herein a “cremophore” is a solvent that permits solubilization of a drug or compound. Various cremophores are well known to one of skill in the art, including but not limited to oil-based solvents.

As used herein “decreasing the size of a tumor” is defined as a reduction in the size of a tumor; the reduction is accomplished by reducing the number of proliferating tumor cells in the tumor, i.e. reducing cell division of the tumor cells, and by inducing cytotoxicity or cell death (apoptosis) of existing tumor cells. Accordingly, tumor growth is arrested or prevented.

As used herein, an “effective amount,” when used with respect to the combination of oxaliplatin and C6-ceramide, includes, without limitation, an amount of oxaliplatin and C6-ceramide which provides the maximum apoptosis of cancer cells at the least toxicity to noncancer cells. The effective amount can be, for example, the concentration of oxaliplatin and ceramide which induces about a 50% death rate (ED 50) of cancer cells. In one example, the instant composition comprises an amount of oxaliplatin which alone would induce an ED 50 of cancer cells, together with an amount of C6-ceramide which alone would induce an ED 50 of cancer cells. In another example, the instant composition comprises at least amounts of oxaliplatin and C6-ceramide which, together, would induce an ED 50 of cancer cells.

As used herein “increasing apoptosis” is defined as an increase in the rate of programmed cell death, i.e. more cells are induced into the death process as compared to exposure (contact with) either oxaliplatin alone or the ceramide alone. Increasing apoptosis also includes the inhibition of cell division which results in a decrease in the total number of viable cancer cells.

As used herein, the term “subject” shall mean any animal including, without limitation, a human, a mouse, a rat, a rabbit, a non-human primate, or any other mammal. In the preferred embodiment, the subject is human. The subject can be male or female.

### Embodiments of the Invention

Applicants demonstrate herein the in vivo anti-tumor effects of combining C6-ceramide with oxaliplatin and cisplatin on the L3.6 human pancreatic adeno-carcinoma implanted in a SCID mouse. Correlative histologic studies provide additional mechanistic insights. This invention provides a method of combination therapy wherein oxaliplatin and ceramide interact synergistically to induce cytotoxicity and apoptosis in carcinoma cells thereby decreasing the growth of cancer cells.

Specifically, this invention provides a method for increasing apoptosis in a cancer cell comprising contacting the cancer cell with (a) oxaliplatin and (b) C6-ceramide, sequentially or concomitantly, wherein the oxaliplatin and C6-ceramide are in amounts such that the apoptosis induced by the combination of oxaliplatin and C6-ceramide is greater than the apoptosis induced by contacting the cancer cell with either oxaliplatin alone or C6-ceramide alone, thereby increasing apoptosis in the cancer cell.

This invention also provides a method of decreasing the size of a tumor, wherein the tumor comprises cancer cells, which method comprises contacting the tumor with (a) oxaliplatin and (b) C6-ceramide, sequentially or concomitantly, wherein the oxaliplatin and C6-ceramide are in amounts such that the decrease in tumor size induced by the combination of oxaliplatin and C6-ceramide is greater than the decrease in tumor size induced by contacting the tumor with either oxaliplatin alone or C6-ceramide alone, thereby decreasing the size of the tumor.

In one embodiment of the above-mentioned methods, the cancer cell (or cancer cells, as applicable) is selected from the group consisting of a leukemic cell, a prostate cancer cell, a pancreatic cancer cell, a squamous cell carcinoma cell, a breast carcinoma cell, a melanoma cell, a basal cell carcinoma cell, a neuroblastoma cell, a glioblastoma multiforme cell, a myeloid leukemic cell, a colon carcinoma cell, an endometrial carcinoma cell, a lung carcinoma cell, an ovarian carcinoma cell, a cervical carcinoma cell, an osteosarcoma cell and a lymphoma cell. In the preferred embodiment, the cancer cell is a pancreatic cancer cell.

In another embodiment of the above-mentioned methods, the cell or tumor is first contacted with oxaliplatin and subsequently contacted with C6-ceramide.

In a further embodiment of the above methods, the cell or tumor is present in a subject.

In another embodiment of the above methods, the contacting with oxaliplatin is effected by cremophore delivery or liposome-mediated delivery, and the contacting with C6-ceramide is effected by cremophore delivery, alcohol-mediated delivery or liposome-mediated delivery.

In another embodiment of the above methods, the contacting with oxaliplatin and with C6-ceramide is effected by an administration route selected from the group consisting of intravenous, intraperitoneal, intrathecal, intralymphatic, intramuscular, intralesional, parenteral, epidural, subcutaneous, pleural, topical, oral, nasal, anal, ocular and otic.

This invention also provides a pharmaceutical composition comprising oxaliplatin, C6-ceramide and a pharmaceutically acceptable carrier, wherein (i) the composition causes apoptosis in a cancer cell, and (ii) the apoptosis induced by the combination of oxaliplatin and C6-ceramide is greater than the apoptosis induced by contacting the cancer cell with either oxaliplatin alone or C6-ceramide alone.

In one embodiment of the above-mentioned pharmaceutical composition, the cancer cell is selected from the group consisting of a leukemic cell, a prostate cancer cell, a pancreatic cancer cell, a squamous cell carcinoma cell, a breast carcinoma cell, a melanoma cell, a basal cell carcinoma cell, a neuroblastoma cell, a glioblastoma multiforme cell, a myeloid leukemic cell, a colon carcinoma cell, an endometrial carcinoma cell, a lung carcinoma cell, an ovarian carcinoma cell, a cervical carcinoma cell, an osteosarcoma cell and a lymphoma cell. In the preferred embodiment, the cancer cell is a pancreatic cancer cell.

Finally, this invention provides a method for treating a subject afflicted with cancer which method comprises administering to the subject (a) oxaliplatin and (b) C6-ceramide, sequentially or concomitantly, wherein the oxaliplatin and C6-ceramide are in amounts such that the apoptosis in the subject's cancer cells induced by the combination of oxaliplatin and C6-ceramide is greater than the apoptosis in the subject's cancer cells induced by contacting the cancer cells with either oxaliplatin alone or C6-ceramide alone, thereby treating the subject afflicted with cancer.

In one embodiment of the above method, the cancer cells are selected from the group consisting of leukemic cells, prostate cancer cells, pancreatic cancer cells, squamous cell carcinoma cells, breast carcinoma cells, melanoma cells, basal cell carcinoma cells, neuroblastoma cells, glioblastoma multiforme cells, myeloid leukemic cells, colon carcinoma cells, endometrial carcinoma cells, lung carcinoma cells, ovarian carcinoma cells, cervical carcinoma cells, osteosarcoma cells and lymphoma cells. In the preferred embodiment, the cancer cells are pancreatic cancer cells.

In another embodiment of the above method, oxaliplatin is first administered and C6-ceramide is subsequently administered to the subject.

In a further embodiment of the above method, C6-ceramide is first administered and oxaliplatin is subsequently administered to the subject.

In further embodiments of the above-described methods and composition, the ceramide may be a C2-ceramide, CS-ceramide, C8-ceramide, C16-ceramide, or a higher order of ceramide. In the preferred embodiment, the ceramide is C6-ceramide. For each embodiment of this invention relating to C6-ceramide, each of the other orders of ceramide listed in this paragraph are also envisioned mutatis mutandis.

In one embodiment of the above methods, the amount of oxaliplatin is from about 1.0 mg/kg-about 3.5 mg/kg every two weeks. In another embodiment, the amount of oxaliplatin is about 2.5 mg/kg every two weeks. In a further embodiment, the amount of oxaliplatin is about 1.5 mg/kg, 2.0 mg/kg or 3.0 mg/kg every two weeks.

In another embodiment of the above methods, the amount of ceramide is from about 1.0 mg/kg-about 10.0 mg/kg every two weeks. In a further embodiment, the amount of ceramide is about 10.0 mg/kg every two weeks. In a further embodiment, the amount of ceramide is about 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10.0 mg/kg, 11.0 mg/kg, 12.0 mg/kg, 13.0 mg/kg, 14.0 mg/kg or 15.0 mg/kg every two weeks. Moreover, all combination permutations of the oxaliplatin and ceramide dosages above are envisioned here.

For each of the above embodiments, the oxaliplatin:ceramide ratio can be, for example, about 1:4.

This invention is illustrated in the Experimental Details section that follows. This section is set forth to aid in an understanding of the instant invention but is not intended to, and should not be construed to, limit in any way the invention as set forth in the claims which follow thereafter.

### EXPERIMENTAL DETAILS

### Materials & Methods

In vitro cytotoxic effects of Paclitaxel, Oxaliplatin and Cisplatin+/−ceramide (C6) were measured by MTT assay. Ceramide 6.25 μg/ml augmented the cytotoxic effects of low dose (subclinical) Paclitaxel 0.06 μg/ml by 3 fold or Paclitaxel 0.6 μg/ml by 1.5 fold. It produced parallel effects on cytoxoxicty induced by low dose cisplatin and oxaliplatin. In vivo experiments utilized SCID/Beige/Taconic male mice inoculated subcutaneously (S.C.) with 2×10⁶ L3.6 pancreatic cells. Chemotherapy dose levels were based on standardized clinical dosing as modified from in vitro data. Treatment began 4 days post tumor implant with thrice weekly (3×/wk) intraperitoneal (IP) injections of paclitaxel (P) (3.0 m/kg), oxaliplatin (OX) (2.5 mg/kg), cisplatin (CP) (0.5 mg/kg), with or without ceramide (10 mg/kg). Mice were observed for 6 weeks and were autopsied when near death, or at the 6 week level. (All controls died by the 3^{rd} week). The data recovered included maximum tumor volume, tumor weight, body weight and survival. Histopathology studies were carried out in a separate group of 40 mice treated by the same drug dose levels and autopsied at 4 hours and 24 hours. Tumors were bi-valved and fixed in buffered formalin or frozen in hexane/acetone bath. A major focus was effects on tumor necrosis, apoptosis, mitotic index and caspase 3 index.

### Cells

The L3.6 is an adherent human pancreatic cell line obtained from the laboratory of Dr. I. Fidler (MD Anderson, Houston, Tex.). It was derived from the L3.3 pancreatic cell line that was originally cultured from a primary pancreatic cancer specimen obtained from a patient previously treated at Roger Williams Medical Center (46).

The L3.6 cells were routinely maintained in T-75 culture flasks (Falcon, N.J.) at a plating cell density of 0.1×10⁶/cm² surface area in complete DMEM/F-12 culture medium (10 ml) containing 10% fetal bovine serum (FBS; Atlanta biologicals, Ga), 2 mM glutamine (Gibco, NY), 50 U/ml penicillin, 50 mg streptomycin (Gibco, NY) and 20 mM HEPES (Sigma, Mo.) at 37° C. in an atmosphere containing 5% CO2. L3.6 cultures were replenished with fresh complete culture medium and reseeded twice weekly.

Treatment of L3.6 Tumors Cells with Paclitaxel and/or Ceramide

Prior to Paclitaxel and/or Ceramide exposure, L3.6 cells were trypsinized in 0.5% trypsin-EDTA, washed twice in complete DMEM/F-12, and plated in 96 well-culture plates at 50×10′ cells/ml in a final volume of 0.2 ml in complete DMEM/F-12. Cells were incubated in the absence or presence of 3 different concentrations of Paclitaxel (0.06 ug/ml, 0.6 ug/ml, and 6.0 ug/ml. Bristol Myers Squibb, NJ) and/or C6-Ceramide (N-hexanoyl-D-sphingosine, 6.25 ug/ml, 12.5 ug/ml and 25 ug/ml. (Sigma Chemicals, MO.) The Paclitaxel concentrations utilized represented a dose range from sub-clinical (0.06 and 0.6 ug/ml) to supraclinical (6.0 ug/ml). Cells were subjected to 1) tetrazolium-based dye assay of survival, b) MTT assay, which was determined at 72 hours (based on previous studies in the laboratory) (27, 28).

### MTT Assay

Cellular cytotoxicity was measured by the addition of 50 ul 0.2% solution of MTT [3-(4,5-dimethylthiazol-2-yl)-2,5-diphtetrazolium bromide] dye (Sigma Chemicals, MO.) to 13.6 cell after treatment with Paclitaxel and/or Ceramide. MTT-treated cultures were then incubated for 4 h at 37° C. Culture plates were centrifuged at 300 g for 2 minutes and the culture supernatants removed. MTT formazin crystals formed by cells undergoing coupled respiration were solubilized by the addition of 150 ul DMSO and subsequent removal of the culture medium Optical density was determined spectrophotometrically (Model EL311, biotek) at 544 nm.

### Study Design

SCID/Beige/Taconic male mice, 22-25 g, 6-8 weeks old purchased from Taconic Laboratory (Germantown, N.Y.) were ear tagged and randomized into eight groups of 5 mice each prior to inoculation s.c. with 2×10⁶ L3.6 PA cells (46) in a volume 0.1 ml into the internal surface of the right thigh. Treatment was started according to the protocol <0.5 cc> (early 4 days or late 10 days). At 4 days after tumor cell injection, mice had developed palpable nubbins of tumor whereas at or 10 days mice had grossly visible tumors chemotherapy 1 cm³ was started. Mice were treated 3 times/week for 4-6 weeks with intraperitoneal injections of chemotherapy at dose levels to be described. Body weight of mice and diameters of tumors were measured every week. Tumor volumes (cm3) were calculated by formula: V=Higher diameter×(smaller diameter)²/2 (47-50). At the end of the 6 week observation period surviving mice were euthanized with CO, inhalation. All the mice were autopsied and tissues were fixed in 10% buffered formalin, embedded in paraffin, and H&E sections of primary tumor, lungs, spleen, liver were examined microscopically. We also analyzed survival rate (%), mean tumor volume (MTV), mean survival time (MST), % mice with primary tumors and final rate of tumor development per survival time (FRTD). The last test was calculated from the MTV and MST: FRTD (cm³/d)=MTV (cm³/MST/day).

### Dosages

The conventional clinical dose levels for oxalplatin are 130 mg/M² every two weeks (equivalent to 3 mg/kg in a prototypic male with BSA of 1.8 M²). The optimum dose level is a function of anti-tumor activity and toxicity. Recommended clinical dose levels as utilized in colorectal studies are:

| | Equivalence of 70 kg (=1.8 M²) |
|---|---|
| 85 mg/M² every 2 wks. | (2.0 mg/kg) |
| 100 mg/M² every 2 wks. | (2.57 mg/kg) |
| 130 mg/M² every 2 wks. | (3.34 mg/kg) |

Our initial dose response studies were done using low dose ceramide 1 mg/kg and oxaliplatin dose levels of 1.0 mg/kg→3.5 mg/kg. Oxaliplatin at 2.5 mg/kg appeared to be most active, and was less toxic than other dose levels. We selected 2.5 mg as the optimum oxaliplatin dose. We next studied the optimum ceramide dose level from 1 mg/kg to 10 mg/kg. Ceramide at 1 mg/kg had only modest effects on enhancing oxaliplatin. Dose level of 10 mg/kg appeared to have optimum synergy regarding anti-tumor effect with lowest toxicity (weight loss). At this point the optimum and most manageable and clinically applicable dose levels appear to be with concurrent systemic injection (intraperitoneal/intervenous) oxaliplatin 2.5 mg/kg and ceramide 10 mg/kg. The dose level for oxaliplatin was based on the in vitro dose response observed in detailed studies with cisplatin. The 72 hour MTT studies focused on cisplatin and demonstrated that cisplatin at dose levels of 1.25 mg/ml reduced cell viability to 65%, which was reduced to 45% with the addition of ceramide. Dose level of 2.5 mg/ml reduced cell viability to 50%, addition of ceramide reduced this to 40%.

Based on the standardized dosing scheme of moderate dose cisplatin at 100 mg/M² and a corresponding molar equivalent dose of oxaliplatin at 30 mg/M², equivalent dose levels are calculated on a mg/kg basis (standardized dose of 100 mg/M² in 70 kg male) with standardized body surface area of 1.8, being equivalent to 180 mg/70 kg 2.57 mg/kg (for cisplatin). A similar calculation for 130 mg/M² of oxaliplatin would be 3.34 mg/kg (for oxaliplatin).

### Results

Combination with C6-ceramide augmented the tumor reduction obtained by chemotherapy alone by 57% (while preserving body weight), and increased 6 week survival from 0% (chemotherapy alone) to 60% with combined therapy. Mean survival was increased from 25 to 37 days. Preliminary short term immunohistochemical studies showed enhancement of apoptotic index and increased, caspase 3 production at 4 and 24 hour by ceramide and the ceramide combinations with (P), (0×), and (CP).

### Effect of C6 Ceramide+/−Chemotherapy on L3-6 Growth in SCID Mice

In Vivo Anti-tumor Response

| | | | | Mean Body |
|---|---|---|---|---|
| | | | | Weight (g) |
| | Mean Final | Mean | % | (Time of |
| | Tumor | Survival | Survival @ | Death |
| Drugs | Volume | Time (days) | 3 & 6 Weeks | or Sacrifice) |
| Control | 1.56 +/− 0.2 | 17.8 +/− 1/1 | 0%/0% | 17.8 |
| Ceramide | 1.69 +/− 0.3 | 20.8 +/− 1.1 | 40%/0% | 17.0 |
| Taxol | 1.83 +/− 0.4 | 23.0 +/− 2.4 | 60%/0% | 17.4 |
| Oxaliplatin | 1.76 +/− 0.2 | 27.4 +/− 2.2 | 100%/0% | 15.6 |
| Cisplatin | 1.83 +/− 0.1 | 25.6 +/− 3.2 | 60%/0% | 16.6 |
| Ceramide & | 1.19 +/− 0.1 | 35.2 +/− 4.0 | 100%/60% | 20.0 |
| Taxol | (++) | (++) | (++) | (++) |
| Ceramide & | 0.75 +/− 0.01 | 35.0 +/− 4.4 | 100%/60% | 20.0 |
| Oxaliplatin | (++) | (++) | (++) | (++) |
| Ceramide & | 1.16 +/− 0.01 | 40.6 +/− 1.4 | 100%/60% | 20.0 |
| Cisplatin | (++) | (++) | (++) | (++) |

Significance
+ p < 0.1,
++ p < 0.05,
+++p < 0.01

### Effect of Ceramide+/−Chemotherapy on L36 Growth in Scid Mice and Histopath Changes 4 hrs/24 hrs

| | Mean | | | | | | |
|---|---|---|---|---|---|---|---|
| | Final | Mean | | % of | | | |
| | Tumor | Survival | % Surviving | Necroses | Mitotic | Apoptosis | Caspase 3 |
| Drugs | Volume | Time | at 3 & 6 wks | 4/24 hr | Index | Index | Index |
| Control | 1.56 +/− 0.2 | 17.8 +/− 1.1 | 0% 0-0% | 20/5 | 1.05 | 0.8/0.8 | 2.65/4.5 |
| Ceramide | 1.69 +/− .03 | 20.8 +/− 1.1 | 40%-0% | 30/NA | 0.6/0/32 | 1.6/1/2 | 4.12/4.25 |
| Taxol | 1.83 +/− 0.4 | 23.0 +/− 2.4 | 60%-0% | 50/NA | 1.5% | 1/4 | 20.5 |
| Oxaliplatin | 1.76 +/− 0.2 | 27.4 +/− 2.2 | 100%-0% | 30%/10%+ | 0.92/0/57+ | 1.35/1.28 | 3.5/3/1 |
| Cisplatin | 1.83 +/− 0.1 | 25.6 +/− 3.2 | 60%-0% | | | | |
| Ceramide & | 1/19 +/− 0.1++ | 35.2 +/− 4.0++ | 100%-60% | 20/15+ | 2.7%/1.8% | 1.65/2.75 | 2.9/7.45+ |
| Taxol | | | | | | | |
| Ceramide & | 0.75 +/− 0.0++ | 35.0 +/− 4.4++ | 100%-60% | 15/30+ | 0.45/0.13%+ | 0.6/0.95 | 4/95/5.05+ |
| Oxaliplatin | | | | | | | |
| Ceramide & | 1.16 +/− 0.0++ | 40.6 +/− ++ | 100%-80% | 10/− | 1.10/ | 1.53 | 3.66 |
| Cisplatin | | | | | | | |
| Taxol & | | | | 30/10 | 0.78/0.38 | 1.55/1.25 | 5.05/4.45 |
| Oxaliplatin | | | | | | | |

Significance +P = <0.1, ++≦0.05

### Tissue Effects of Short Term Treatment (4/24 hrs) with Drug+/−Ceramide

Tissue examination for apoptosis by conventional H & E exam, and by staining with antibody to caspase 3 was carried out in mice bearing L36 Tumors—4 hr & 24 hours after treatment with Paclitaxel+/−Ceramide and Oxaliplatin+/−Ceramide, and Paclitaxel+Oxaliplatin+/−Ceramide and Ceramide alone.

### (Difference in Caspase Expression)

| | Apoptosis | Caspase | | Vs. Drug & |
|---|---|---|---|---|
| | N/4000 Cells | N/4000 Cells | Vs. Control | Ceramide |
| | 4/24 hrs | 4/24 hrs | 4/24 hrs | 4/24 hrs |
| Control | 32/32 | 106/108 | | |
| 4/24 hrs | | | | |
| Ceramide | 64/46 | 165/157 | 155%/148% | |
| Paclitaxel | 66/110 | 101/ | 95%/ | |
| Paclitaxel & | 66/110 | 117/298 | 110%/281% | 110%/295% |
| Ceramide | | | | |
| Oxaliplatin | 54/51 | 148/123 | 132%/121% | |
| Oaxilplatin & | 24/38 | 196/202 | 184%/190% | 140%/165% |
| Ceramide | | | | |
| Paclitaxel & | 62/45 | 205/178 | 193%/168% | |
| Oxaliplatin | | | | |
| Pac & Oxal & | 61/ | 147 | 71% | 71%/82% |
| Cer | | | | |

Pilot data suggest value in measuring caspase expression to determine drug effects on apoptosis.

### CONCLUSION

Combination therapy with the apoptotic signal C6-ceramide significantly enhanced the anti-tumor response to Paclitaxel, Oxaliplatin and Cisplatin in SCID Mice bearing L3.6 pancreatic tumor implants with preservation of animal weight. Early histologic evidence enhanced apoptosis and caspase 3 expressions are suggested in preliminary short term exposure experiments.

### REFERENCES

1. Cifone, M. G., De Maria, R., Roncaioli, P., Rippo, M. R., Azuma, M., Lanier, L. L., Santoni, A., Testi, R., Apoptotic signalling through CD95 (Fas/Apo-1) activates an acidic sphingomyelinase. J Exp Med., 1994, 180, 1547.
Testi, R. Sphingomyelin breakdown and cell fate. Trends in Biochem Sci, 1996, 21, 468.
3. Jarvis, W. D., Grant, S., and Kolesnick, R. N., Ceramide and the induction of apoptosis. Clin Cancer Res., 1996 2, 1.
4. Obeid, L. M., Hannun, Y. A., Ceramide: a stress signal and mediator of growth supression and apoptosis. J Cell Biochem., 1995, 58, 191.
5. Obeid, L. M., Linardic, C. M., Karolak, L. A., Hannun, Y. A., Programmed cell death induced by ceramide. Science, 1993, 259, 1769.
Ji, L., Zhang, G., Uematsu, S., Akahori, Y., Hirabayashi, Y., Induction of apoptotic DNA fragmentation and cell death by natural ceramide. FEBS Letters, 1995, 358, 211.
7. Hannun, Y. A., Obeid, L. M., Ceramide: an intracellular signal for apoptosis. Trends in Biochem Sci., 1995, 20, 73.
8. Tepper, C. G., Jayadev, S., Liu, B., Bielawska, A., Wolff, R., Yonehara, S., Hannun, Y. A., Seldin, M. F., Role for ceramide as an endogenous mediator of Fas-induced cytotoxicity. Proc. Natl. Acad. Sci., 1995, 92, 8443.
9. Kolesnick, R. N., Haimovitz-Friedman, A., Fuks, Z., The sphingomyelin signal transduction pathway mediates apoptosis for tumor necrosis factor, Fas, and ionizing radiation. Biochem. and Cell Biol., 1994, 72, 471.
10. Jarvis, W. D., Kolesnick, R. N., Fornari, F. A., Traylor, R. S., Gewirtz, D. A., and Grant, S., Induction of apoptotic DNA damage and cell death by activation of the sphingomyelin pathway. Proc. Natl. Acad. Sci., 1994, 91, 73.
11, Kuroki, J., Hirokawa, M., Kitabayashi, A., Lee, M., Horiuchi, T., Kawabata, Y., Miura, A. B., Cell-permeable ceramide inhibits growth of B lymphoma Raji cells lacking TNF-alpha receptors by inducing G₀/G₁ arrest but not apoptosis: a new model for dissecting cell-cycle arrest and apoptosis. Leukemia 1996, 10, 1950.
12. Jayadev, S., Liu, B., Bielawska, A. E., Lee, J. Y., Nazaire, F., Pushkareva, M. Yu, Obeid, L. M., Hannun, Y. A., Role for ceramide in cell cycle arrest. J. Biol. Chem., 1995, 270, 2047.
13. Venable, M. E., Lee, J. Y., Smyth, M. J., Bielawska, A., Obeid, L. M., Role of Ceramide in Cellular Senescence. J. Biol. Chem., 1995, 270, 30701.
14. Hannun, Y. A. The sphingomyelin cycle and the second messenger function of ceramide. J. Biol. Chem., 269, 3125, 1994.
15. Kolesnick, R. and Golde, D. W. The sphingomyelin pathway in tumor necrosis factor and interleukin-1 signaling. Cell, 77, 325, 1994.
16. Ballou, L. R., Chao, C. P., Holness, M. A., Barker, S. C., and Raghow, R. Interleukin-1-mediated PGE2 production and sphingomyelin metabolism. Evidence for the regulation of cyclooxygenase gene expression by sphingosine and ceramide. J. Biol. Chem., 267, 20044, 1992.
17. Yanaga, F. and Watson, S. P. Ceramide does not mediate the effect of tumour necrosis factor alpha on superoxide generation in human neutrophils. Biochem. J., 298, 733, 1994.
18. Okazaki, T., Bielawska, A., Bell, R. M., and Hannun, Y. A. Role of ceramide as a lipid mediator of 1 alpha, 25-dihydroxyvitamin D3-induced HL-60 cell differentiation. J. Biol. Chem., 265, 15823, 1990.
19. Dobrowaky, R. T., Jenkins, G. M., and Hannun, Y. A. Neurotrophins induce sphingomyelin hydrolisis. Modulation by co-expression of p75NTR with Trk receptors. J. Biol. Chem., 270, 22135, 1995.
20. Venable, M. E., Lee, J. Y., Smyth, M. J., Bielawska, A, Obeid, L. M. Role of ceramide in cellular senecence. J. Biol. Chem., 270, 30701, 1995.
21. Bose, R., Verheji, M., Haimovitz-Friedman, A., Scotto, K., Fuks, Z. and Kolesnick, R. Ceramide synthase mediates daunorubicin-induced apoptosis: and alternative mechanism for generating death signals. Cell, 82: 405-414, 1995.
22. Strum, J. C., Small, G. W., Daiug, S, and Daniel, L. W., 1-b-D arabinofuranosylcytosine stimulates ceramide and diglyceride formation in HL-60 cells. J. Biol. Chem., 269, 15493, 1994.
23. Jayadev, S., Liu, B. Bielawska, A. E., Lee, J. Y., Nazaire, F., Pushkareva, M., Obeid, L. M. and Hannun, Y. A. Role for ceramide in cell cycle arrest. J. Biol. Chem., 270, 2047, 1995.
24. Beilawska, A., Linardic, C. M., and Hannun, Y. A. Modulation of cell growth and differentiation by ceramide. FEBS Lett, 307, 211, 1992.
25. Obeid, L. M., Hannun, Y. A. Ceramide: a stress signal and mediator of growth supression and apoptosis. J. Cell Biochem., 58, 191, 1995.
26. Jarvis, W. D., Kolesnick, R. N., Fornari, F. A., Traylor, R. S., Gewirtz, D. A., and Grant, S. Induction of apoptotic DNA damage and cell death by activation of the sphingomyelin pathway. Proc. Natl. Acad. Sci USA, 91, 73, 1994.
27. Elion, G B, Singer, S and Hichings G H. Antagonists of nucleic acid derivatives. VIII. Synergism in combinations of biochemically related antimetabolites. J. Biol. Chem., 208, 477, 1954.
28. Chou, T. C. and Talalay P. Quantitative analysis of dose-effect relationships: the combined effect of multiple drugs and enzyme inhibitors. In: Advances in enzyme regulation. G. Weber, ed, Pergamon Press, NY, pp 27-55, 1984.
29. Hannun, Y. Functions of ceramide in coordinating cellular responses to stress. Science, 274, 1855, 1996.
30. Sweeney, E, Sakakura, C., Shirahama, T., Masamune, A., Ohta, H., Hakomori, S, and Igarashi, Y. Sphingosine and its methylated derivative N,N-dimethyl sphingosine (DMS) induce apoptosis in a variety of human cancer cell lines. Int. J. Cancer, 66, 358, 1996.
31. Dressler, K. A., Mathias, S, and Kolesnick, R. N. Tumor necrosis factor-alpha activates the sphingomyelin signal transduction pathway in a cell-free system. Science, 255, 1715, 1992.
32. Kim, M. Y. Identification of sphingomyelin turnover as an effector mechanism for the action of tumor necrosis factor alpha and gamma-interferon. Specific role in cell differentiation. J. Biol. Chem., 266, 484, 1991.
33. Gulbins, E., Bissonette, R., Mahboudi, A., Martin, S., Nishioka, W., Brunner, T., Baier, G., Baier-Bitterlich G., Lang F. et al. FAS-induced apoptosis is mediated via a ceramide-initiated RAS signaling pathway. Immunity, 2, 341, 1995.
34. Kerr, J. F., Wyllie, A. B., and Currie, A. R. Apoptosis: a basic biological phenomenon with wide ranging implications in tissue kinetics. Br. J. Cancer, 26, 239, 1972.
35. Bursh, W., Kliene, L., and Tenniswood, M. The biochemistry of cell death by apoptosis. Biochem. Cell, Biol., 68, 1071, 1990.
36. Friesen C., Herr, I., Krammer, P H and Debatin K M. Involvement of the CD95 (APO-1/PAS) receptor/ligand system in drug-induced apoptosis in leukemia cells. Nature Medicine, 2, 574, 1996.
37. Villunger, A., Egle, A., Kos, M., Hartmann B L, Geley S, Kofler R and Grell R. Drug-induced apoptosis is associated with enhanced Fas (Apo-1/CD95) ligand expression but occurs independently of Pas (Apo-1/CD95) signaling in human T-acute lymphatic leukemia cells. Cancer Res., 57, 3331, 1997.
38. Eischen, C. M., Kottke, T. J. Martins L M, Basi, G. S., Tung J. S., Earnshaw, W. C., Liebson P J and Kaufmann S H. Comparison of apoptosis in wild-type and Fas-resistant cells: chemotherapy-induced apoptosis is not dependent on Fas/Fas ligand interactions. Blood, 90, 935-43, 1997.
39. Bielawska A, Linadic C M, Hannun Y A. Modulation of cell growth and differentiation by Ceramide, FEES Lett. 307; 211, 1992.
40. Kolesnick R N, Kronke N. Regulation of Ceramide production and apoptosis. Annu Rev Physiol 60:643-64, 1998.
41. Myrick D, Blackinton D, Klostergaard N, Maizel A, Wanebo H J, Mehta S. Paclitaxel Induced apoptosis in Jurkat, a leukemic T-cell line, is enhanced by Ceramide. Leuk. Res. 23:569-78, 1999.
42. Senchenkovic A, Litvak D A, Cabot M C. Targeting Ceramide metabolism-strategy for overcoming drug resistance a review. J Ntl Ca Inst 93:347-57, 2001.
43, Bose R, Verheji M, Haimovitz-Freidman A, Soctto K, Fuks Z, Kolesnick R. Ceramide Synthase mediates Daunorubicin-induced apoptosis; an alternative mechanism for generating death signal. Cell 82:405-14, 1995.
44. Lucci A, Han T Y, Liu Y Y, Giuliano A E, Cabot M C. Multi-drug resistance Modulators and doxorubicin synergize to elevate Ceramide levels and elicit apoptosis in drug resistance cancer cells. Cancer; 82:30-11, 1999.
45. Charles A G, Han T Y, Liu U U, Hanse N, Giuliano A E, Cabot M C. Paclitaxel-induced Ceramide generation and apoptosis in human breast cancer cells. Cancer Chemothr Pharmacol; 47(5): 444-50, 2001.
46. Seidler, M. et al. (1995) “Characterization of human pancreatic adenocarcinoma cell line with high metastatic potential in SCID mice,” Invasion Metastasis 15: 160-169.
47. Dommilen, J. I. et al. (2003) “Activation of natural killer (NK) T cells during murine cytomegalovirus infection enhances the antiviral repair mediated by NK cells,” J. Viral, 77:3, 1877-1884.
48, Toura, I. et al. (1999) “Catting edge: inhibition of experimental tumor metastasis by dendritic cells pulsed with alpha-GalactosylCeramide,” J. Immunol. 163: 2387-2391.
49. Nakagawa, R. et al. (2000) “Antitumor activity of alpha-galactosylCeramide, KRN 7000, in mice with the melanoma B16,” Oncol. Res. 12(2): 51-8.
50. Kikuchi, A. et al. (2001) “In vitro anti-tumor activity of alpha-galactoCeramide-stimulated human variant Vα24+NKT cells against melanoma,” Brit. J. Cancer 85:5, 741-746.

1. A method for increasing apoptosis in a tumor comprising pancreatic cancer cells which method comprises contacting the tumor with (a) an amount of oxaliplatin and (b) an amount of C6-ceramide, sequentially or concomitantly, wherein the amount of oxaliplatin and the amount of C6-ceramide in combination is effective to induce apoptosis of the pancreatic cells, wherein the apoptosis of the pancreatic cells is greater than the apoptosis induced by contacting the pancreatic cancer cells with either the amount of oxaliplatin alone or the amount of C6-ceramide alone, thereby increasing apoptosis in the tumor.

2. (canceled)

3. (canceled)

4. The method of claim 1, wherein the tumor is first contacted with the amount of oxaliplatin and subsequently contacted with the amount of C6-ceramide.

5. The method of claim 1, wherein the tumor is present in a subject.

6. The method of claim 1, wherein the contacting with the amount of oxaliplatin is effected by cremophore delivery or liposome-mediated delivery, and the contacting with the amount C6-ceramide is effected by cremophore delivery, alcohol-mediated delivery or liposome-mediated delivery.

7. The method of claim 1, wherein the contacting with the amount of oxaliplatin and with the amount of C6-ceramide is effected by an administration route selected from the group consisting of intravenous, intraperitoneal, intrathecal, intralymphatic, instramuscular, instralesional, parenteral, epidural, subcutaneous, pleural, topical, oral, nasal, anal, ocular and otic.

8. A method of decreasing the size of a tumor, wherein the tumor comprises pancreatic cancer cells, which method comprises contacting the tumor with (a) an amount of oxaliplatin and (b) an amount of C6-ceramide, sequentially or concomitantly, wherein the amount of oxaliplatin and the amount of C6-ceramide in combination is effective to decrease the size of the tumor, wherein the decrease in size of the tumor is greater than the decrease in tumor size induced by contacting the tumor with either the amount of oxaliplatin alone or the amount of C6-ceramide alone, thereby decreasing the size of the tumor.

9. (canceled)

10. (canceled)

11. The method of claim 1, wherein the tumor is first contacted with the amount of oxaliplatin and subsequently contacted with the amount of C6-ceramide.

12. The method of claim 8, wherein the tumor is present in a subject.

13. The method of claim 8, wherein the contacting with the amount of oxaliplatin is effected by cremophore delivery or liposome-mediated delivery, and the contacting with the amount of C6-ceramide is effected by cremophore delivery, alcohol-mediated delivery or liposome-mediated delivery.

14. The method of claim 8, wherein the contacting with the amount of oxaliplatin and with the amount of C6-ceramide is effected by an administration route selected from the group consisting of intravenous, intraperitoneal, intrathecal, intralymphatic, intramuscular, intralesional, parenteral, epidural, subcutaneous, pleural, topical, oral, nasal, anal, ocular and otic.

15. A pharmaceutical composition comprising an amount of oxaliplatin, an amount of C6-ceramide and a pharmaceutically acceptable carrier, wherein (i) the composition causes apoptosis in a tumor comprising pancreatic cancer cells, and (ii) the apoptosis induced by the combination of the amount of oxaliplatin and the amount of C6-ceramide is greater than the apoptosis induced by contacting the tumor with either the amount of oxaliplatin alone or the amount of C6-ceramide alone.

16. (canceled)

17. (canceled)

18. A method of treating a subject afflicted with pancreatic cancer, wherein the pancreatic cancer comprises pancreatic cancer cells, which method comprises administering to the subject (a) an amount of oxaliplatin and (b) an amount of C6-ceramide, sequentially or concomitantly, wherein the amount of oxaliplatin and the amount of C6-ceramide in combination is effective to induce apoptosis of the pancreatic cells, wherein the apoptosis of the pancreatic cells is greater than the apoptosis in the subject's pancreatic cancer cells induced by contacting the pancreatic cancer cells with either the amount of oxaliplatin alone or the amount of C6-ceramide alone, thereby treating the subject afflicted with pancreatic cancer.

19. (canceled)

20. (canceled)

21. The method of claim 18, wherein the amount of oxaliplatin is first administered and the amount of C6-ceramide is subsequently administered to the subject.

22. The method of claim 18, wherein the amount of C6-ceramide is first administered to the subject and the amount of oxaliplatin is subsequently administered to the subject.
